(19) United States
(12) Patent Application Publication
(10) Pub. No.: US 2024/0009221 A1
(43) Pub. Date: Jan. 11, 2024

(54) PHARMACOLOGIC APPROACH FOR SUPPRESSION OF CORONAVIRUSES

(71) Applicant: Temple University - of the Commonwealth System of Higher Education, Philadelphia, PA (US)

(72) Inventors: Kamel Khalili, Bala Cynwyd, PA (US); Shuren Liao, Philadelphia, PA (US); Ilker K. Sariyer, Broomall, PA (US)

(21) Appl. No.: 18/300,468

(22) Filed: Apr. 14, 2023

Related U.S. Application Data
(63) Continuation of application No. PCT/US2021/054949, filed on Oct. 14, 2021.
(60) Provisional application No. 63/091,452, filed on Oct. 14, 2020.

(51) Int. Cl. A61K 31/706; A61K 31/505; A61P 31/14

(52) U.S. Cl. CPC A61K 31/706; A61K 31/505; A61P 31/14

(57) ABSTRACT

Compositions for prevention or treatment for the SARS family of coronaviruses, e.g., SARS-CoV-2 and SARS-OC43 (common cold virus), include non-nucleoside reverse transcriptase inhibitors (NNRTI), such as rilpivirine, and nucleoside-analogue antiviral agents, such as remdesivir.

### CROSS REFERENCE TO RELATED APPLICATIONS

This application claims the benefit of U.S. Provisional Application 63/091,452 filed on Oct. 14, 2020. The entire contents of this application is incorporated herein by reference in its entirety.

### TECHNICAL FIELD

The present disclosure relates to a preventive treatment of diseases caused by coronaviruses, such as pandemic COVID 19, and a therapeutic treatment which includes one or more pharmacological anti-viral agents.

### BACKGROUND

Coronaviruses are enveloped, positive-sense single-stranded RNA viruses. They have the largest genomes (26-32 kb) among known RNA viruses, and are phylogenetically divided into four genera (α, β, γ, δ), with beta coronaviruses further subdivided into four lineages (A, B, C, D). Coronaviruses infect a wide range of avian and mammalian species, including humans. Of the six known human coronaviruses, four of them (HCoV-OC43, HCoV-229E, HCoV-HKU1 and HCoV-NL63) circulate annually in humans and generally cause mild respiratory diseases, although severity can be greater in infants, elderly, and the immunocompromised. In contrast, the Middle East respiratory syndrome coronavirus (MERS-CoV) and the severe acute respiratory syndrome coronavirus (SARS-CoV), belonging to beta coronavirus lineages C and B, respectively, are highly pathogenic. Both viruses emerged into the human population from animal reservoirs within the last 15 years and caused outbreaks with high case-fatality rates.

SARS CoV 2 is the virus responsible for COVID 19, the pandemic disease initiated in Wuhan. China. It provokes severe acute respiratory syndromes, that may lead to death (Yang et al., Cellular & Molecular Immunology; doi.org/10.1038/s41423-020-0407-x). The high pathogenicity and airborne transmissibility of SARS-CoV and MERS-CoV, the high case-fatality rate, vaguely defined epidemiology, and absence of prophylactic or therapeutic measures against coronaviruses have created an urgent need for an effective vaccine and related therapeutic agents.

### SUMMARY

Embodiments are directed to pharmacological compositions for the prevention and treatment of coronavirus infections inclusive of severe acute respiratory syndrome coronavirus (SARS-CoV), SARS-OC43 (common cold virus), MERS (Middle East respiratory syndrome coronavirus).

In certain embodiments, the compositions comprise a therapeutically effective amount of one or more anti-viral agents. In certain embodiments, the compositions comprise a therapeutically effective amount of one or more non-nucleoside reverse transcriptase inhibitors (NNRTI). In certain embodiments, the NNRTI comprises a therapeutically effective amount of one or more of: etravirine, efavirenz, nevirapine, rilpivirine, delavirdine, or nevirapine. In certain embodiments, the NNRTI is rilpivirine. In certain embodiments, the compositions comprise a therapeutically effective amount of one or more nucleoside-analogue antiviral agents. In certain embodiments, the nucleoside-analogue antiviral agent comprises a therapeutically effective amount of one or more of remdesivir, ribavirin, lamivudine, stavudine or zidovudine. In certain embodiments, the nucleoside-analogue antiviral agent is remdesivir.

In certain embodiments, the compositions comprise a therapeutically effective amount of one or more non-nucleoside reverse transcriptase inhibitors (NNRTI) and one or more nucleoside-analogue antiviral agents. In certain embodiments, the compositions comprise a therapeutically effective amount of rilpivirine and remdesivir.

In certain embodiments, a method of treating a coronavirus comprises administering to a subject a composition comprising a therapeutically effective amount of one or more anti-viral agents. In certain embodiments, the compositions comprise a therapeutically effective amount of one or more non-nucleoside reverse transcriptase inhibitors (NNRTI). In certain embodiments, the NNRTI comprises a therapeutically effective amount of one or more of: etravirine, efavirenz, nevirapine, rilpivirine, delavirdine, or nevirapine. In certain embodiments, the NNRTI is rilpivirine. In certain embodiments, the compositions comprise a therapeutically effective amount of one or more nucleoside-analogue antiviral agents. In certain embodiments, the nucleoside-analogue antiviral agent comprises a therapeutically effective amount of one or more of remdesivir, ribavirin, lamivudine, stavudine or zidoduvine. In certain embodiments, the nucleoside-analogue antiviral agent is remdesivir. In certain embodiments, the compositions comprise a therapeutically effective amount of one or more non-nucleoside reverse transcriptase inhibitors (NNRTI) and one or more nucleoside-analogue antiviral agents. In certain embodiments, the compositions comprise a therapeutically effective amount of rilpivirine and remdesivir.

In certain embodiments, the one or more anti-viral agents are co-administered with one or more secondary active agents. Secondary active agents may include protease inhibitors, e.g., tipiravir, darunavir, indinavir; entry inhibitors, e.g., maraviroc; fusion inhibitors, e.g., enfuviritide; or integrase inhibitors e.g., raltegrivir, dolutegravir. The agents may also include multi-class combination agents for example, combinations of emtricitabine, efavarenz, and tenofivir; combinations of emtricitabine; rilpivirine, and tenofivir; or combinations of elvitegravir, cobicistat, emtricitabine and tenofivir.

In certain embodiments, a recombinant vector comprises a nucleic acid sequence encoding a coronavirus protein comprising a spike (S) protein, a membrane (M) protein, an envelope (E) protein or a nucleocapsid (N) protein or immunogenic fragments thereof. In certain embodiments, the coronavirus comprises severe acute respiratory syndrome coronavirus (SARS-CoV-2), SARS-OC43, middle east respiratory syndrome coronavirus (MERS-CoV), human coronavirus (HCoV)-OC43, HCoV-229E, ICoV-HKU1 or HCoV-N L63. In certain embodiments, the coronavirus is a severe acute respiratory syndrome coronavirus (SARS-CoV-2). In certain embodiments, the coronavirus is SARS-OC43.

In certain embodiments, a method of preventing or treating a coronavirus infection, comprises administering to a subject in need of such treatment a composition comprising a therapeutically effective amount of one or more anti-viral agents, and, a recombinant vector comprising a nucleic acid sequence encoding a coronavirus protein comprising a spike (S) protein, a membrane (M) protein, an envelope (E) protein or a nucleocapsid (N) protein or immunogenic fragments thereof, thereby treating the subject. In certain embodiments, the wherein the composition comprises a therapeutically effective amount of one or more of: non-nucleoside reverse transcriptase inhibitors (NNRTI), nucleoside-analogue antiviral agents or a combination thereof. In certain embodiments, the NNRTI comprises a therapeutically effective amount of one or more of: etravirine, efavirenz, nevirapine, rilpivirine, delavirdine, or nevirapine. In certain embodiments, the NNRTI is rilpivirine. In certain embodiments, the nucleoside-analogue antiviral agent comprises a therapeutically effective amount of one or more of remdesivir, ribavirin, lamivudine, stavudine or zidoduvine. In certain embodiments, the nucleoside-analogue antiviral agent is remdesivir. In certain embodiments, the composition comprises a therapeutically effective amount of rilpivirine and remdesivir.

In still yet another embodiment, a packaged device or kit is provided that includes the pharmaceutical composition described herein optionally together with instructions for use. In one embodiment, the device is selected from the group consisting of aerosol dispenser, pneumatically pressurized device, multi-dose metered dose spray pump, inhaler, pump sprayer, and nebulizer.

Other aspects are discussed infra.

### Definitions

The terminology used herein is for the purpose of describing particular embodiments only and is not intended to be limiting of the invention. Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the term “secondary active agent”, refers to any molecule that is used for the prevention or treatment of a virus infection. e.g. a respiratory virus infection, and include agents which alleviate any symptom, such as runny nose, blocked nose, sore throat, sneezing, chilliness, headache, muscle ache, cough, etc., associated with the virus. The term is inclusive of agents which alleviate any symptoms associated with the virus, for example, anti-pyretic agents, anti-inflammatory agents, chemotherapeutic agents, and the like. The “secondary active agent” can be a compound that is directly or indirectly effective in specifically interfering with at least one viral action, such as for example, virus penetration of eukaryotic cells, virus replication in eukaryotic cells, virus assembly, virus release from infected eukaryotic cells, or that is effective in inhibiting a virus titer increase or in reducing a virus titer level in a eukaryotic or mammalian host system. It also refers to a compound that prevents from or reduces the likelihood of getting a viral infection. The term includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, nucleoside reverse transcriptase inhibitors (NRTIs), analogs, variants etc. or combinations thereof.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “effective dose” or “effective dosage” is defined as an amount sufficient to achieve or at least partially achieve the desired effect.

As used herein, the term “fragment,” when referring to a protein or nucleic acid, for example, means any shorter sequence than the full-length protein or nucleic acid. Accordingly, any sequence of a nucleic acid or protein other than the full-length nucleic acid or protein sequence can be a fragment. In some aspects, a protein fragment includes an epitope. In other aspects, a protein fragment is an epitope.

The term “immunogenic fragment” as used herein refers to a polypeptide or a fragment of a polypeptide, or a nucleotide sequence encoding the same which comprises an allele-specific motif, an epitope or other sequence such that the polypeptide or the fragment will bind an MHC molecule and induce a cytotoxic T lymphocyte (“CTL”) response, and/or a B cell response (for example, antibody production), and/or T-helper lymphocyte response, and/or a delayed type hypersensitivity (DTH) response against the antigen from which the immunogenic polypeptide or the immunogenic fragment is derived. A DTH response is an immune reaction in which T cell-dependent macrophage activation and inflammation cause tissue injury. A DTH reaction to the subcutaneous injection of antigen is often used as an assay for cell-mediated immunity.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used herein, the term “nucleic acid” refers to any deoxyribonucleic acid (DNA) molecule, ribonucleic acid (RNA) molecule, or nucleic acid analogues. A DNA or RNA molecule can be double-stranded or single-stranded and can be of any size. Exemplary nucleic acids include, but are not limited to, chromosomal DNA, plasmid DNA. cDNA, cell-free DNA (cfDNA), mitochondrial DNA, chloroplast DNA, viral DNA, mRNA, tRNA, rRNA, long non-coding RNA, siRNA, micro RNA (miRNA or miR), hnRNA, and viral RNA. Exemplary nucleic analogues include peptide nucleic acid, morpholino- and locked nucleic acid, glycol nucleic acid, and threose nucleic acid. As used herein, the term “nucleic acid molecule” is meant to include fragments of nucleic acid molecules as well as any full-length or non-fragmented nucleic acid molecule, for example. As used herein, the terms “nucleic acid” and “nucleic acid molecule” can be used interchangeably, unless context clearly indicates otherwise.

As used in this specification and the appended claims, the term “or” is generally employed in its sense including “and/or” unless the content clearly dictates otherwise.

The term “patient” includes human and other mammalian subjects that receive either prophylactic or therapeutic treatment.

As used herein, the term “protein” refers to any polymeric chain of amino acids. The terms “peptide” and “polypeptide” can be used interchangeably with the term protein, unless context clearly indicates otherwise, and can also refer to a polymeric chain of amino acids. The term “protein” encompasses native or artificial proteins, protein fragments and polypeptide analogs of a protein sequence. A protein may be monomeric or polymeric. The term “protein” encompasses fragments and variants (including fragments of variants) thereof, unless otherwise contradicted by context.

The term “prophylactic treatment” as used herein, refers to any intervention using the compositions embodied herein, that is administered to an individual in need thereof or having an increased risk of acquiring a respiratory tract infection, wherein the intervention is carried out prior to the onset of a viral infection, e.g. SARS-CoV-2, and typically has in effect that either no viral infection occurs or no clinically relevant symptoms of a viral infection occur in a healthy individual upon subsequent exposure to an amount of infectious viral agent that would otherwise, i.e. in the absence of such a prophylactic treatment, be sufficient to cause a viral infection.

The term “therapeutically effective dose” is defined as an amount sufficient to cure or at least partially arrest the disease, e.g. COVID-19 and its complications in a patient already suffering from the disease.

The term “therapy” or “therapeutic treatment” as used herein relates to the administration of the compositions embodied herein, e.g. rilpivirine and/or remdesivir, in order to achieve a reduction in severity and/or frequency of symptoms, elimination of symptoms and/or underlying cause, prevention of the occurrence of symptoms and/or their underlying cause, and/or improvement or remediation of damage directly caused by or indirectly associated, e.g. through secondary infection, with the viral infection.

“Treatment”, or “treating” as used herein, is defined as the application or administration of a therapeutic agent or combination of therapeutic agents (e.g., rilpivirine and/or remdesivir optionally, another active agent, e.g. anti-viral agent preventing agent) to a patient, or application or administration of the active agent to a patient, who has a virus infection, e.g. SARS-CoV-2 with the purpose to cure, heal, alleviate, relieve, alter, remedy, ameliorate, improve or affect the infection, or symptoms thereof. The term “treatment” or “treating” is also used herein in the context of administering agents prophylactically. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof; or (4) relieving the disease, i.e., causing regression of the state, disorder or condition or at least one of its clinical or subclinical symptoms. The benefit to an individual to be treated is either statistically significant or at least perceptible to the patient or to the physician.

The term “vector,” or “recombinant vector” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”. Vectors, including expression vectors, comprise the nucleotide sequence encoding the antibodies or antigen-binding fragments described herein and a heterogeneous sequence necessary for proper propagation of the vector and expression of the encoded polypeptide. The heterogeneous sequence (i.e., sequence from a difference species than the polypeptide) can comprise a heterologous promoter or heterologous transcriptional regulatory region that allows for expression of the polypeptide. As used herein, the terms “heterologous promoter,” “promoter,” “promoter region.” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains (consensus sequences) responsible for the binding of RNA polymerase.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

FIG. 1 is a graph demonstrating that remdesivir and rilpivirine suppressed human coronavirus OC43 replication in a dose dependent manner. Vero E6 cells were infected with OC43 (MOI:1). Cells were treated with increasing concentrations of remdesivir and rilpivirine (0.1, 1.0, and 10 μM). At 48 hrs post-infections, OC43 RNA copies in the growth media were analyzed by real time qRT-PCR with nucleocapsid (N) primers and shown as bar graph. Data were mean±SEM of three replicates. “P” values were calculated in comparison with control infections (untreated). (Inset) Superimposition of OC43 (model) and SARS-CoV-2 (cryo-EM) RdRp structures.

### DETAILED DESCRIPTION

RNA dependent RNA polymerase (RDRP) plays a crucial role in the replication of coronaviruses including SARS. The disclosure is based on the finding that rilpivirine and/or remdesivir alone or in combination can be used as a therapeutic and/or protective strategy in the treatment of infections by the SARS family of coronaviruses, including SARS-CoV-2 and SARS-OC43 (common cold virus).

Accordingly, embodiments are directed to pharmaceutical agents which inhibit viral replication, such as, for example, by inhibiting viral RNA-dependent RNA polymerases.

In certain embodiments, a method of treating a subject in need thereof, comprises administering to the subject a composition comprising: a therapeutically effective amount of one or more of: non-nucleoside reverse transcriptase inhibitors (NNRTI), nucleoside-analogue antiviral agents or a combination thereof. In certain embodiments, an NNRTI comprises: etravirine, efavirenz, nevirapine, rilpivirine, delavirdine, or nevirapine. In embodiments, an NRT1 comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In certain embodiments, the nucleoside-analogue antiviral agent comprises a therapeutically effective amount of one or more of remdesivir, ribavirin, lamivudine, stavudine or zidoduvine.

In certain embodiments, a method of treatment further comprises administering one or more secondary active agents in conjunction with the compositions, e.g. rilpivirine and/or remdesivir, such as, viral entry inhibitors, reverse transcriptase inhibitors, protease inhibitors, and immune-based therapeutic agents.

In certain embodiments, the secondary active agents comprise therapeutically effective amounts of: antibodies, aptamers, adjuvants, anti-sense oligonucleotides, chemokines, cytokines, immune stimulating agents, immune modulating molecules. B-cell modulators, T-cell modulators, NK cell modulators, antigen presenting cell modulators, enzymes, siRNA's, interferon, ribavirin, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

The immune-modulating molecules comprise, but are not limited to cytokines, lymphokines, T cell co-stimulatory ligands, etc. An immune-modulating molecule positively and/or negatively influences the humoral and/or cellular immune system, particularly its cellular and/or non-cellular components, its functions, and/or its interactions with other physiological systems. The immune-modulating molecule may be selected from the group comprising cytokines, chemokines, macrophage migration inhibitory factor (MIF; as described, inter alia, in Bernhagen (1998), Mol Med 76(3-4); 151-61 or Metz (1997). Adv Immunol 66, 197-223), T-cell receptors or soluble MHC molecules. Such immune-modulating effector molecules are well known in the art and are described, inter alia, in Paul, “Fundamental immunology”, Raven Press, New York (1989). In particular, known cytokines and chemokines are described in Meager, “The Molecular Biology of Cytokines” (1998), John Wiley & Sons, Ltd., Chichester, West Sussex, England; (Bacon (1998). Cytokine Growth Factor Rev 9(2):167-73; Oppenheim (1997). Clin Cancer Res 12, 2682-6; Taub, (1994) Ther. Immunol. 1(4), 229-46 or Michiel, (1992). Semin Cancer Biol 3(1), 3-15).

Immune cell activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

Also of interest are enzymes present in the lytic package that cytotoxic T lymphocytes or LAK cells deliver to their targets. Perforin, a pore-forming protein, and Fas ligand are major cytolytic molecules in these cells (Brandau et al., Clin. Cancer Res. 6:3729, 2000; Cruz et al., Br. J. Cancer 81:881, 1999). CTLs also express a family of at least 11 serine proteases termed granzymes, which have four primary substrate specificities (Kam et al., Biochim. Biophys. Acta 1477:307, 2000). Low concentrations of streptolysin O and pneumolysin facilitate granzyme B-dependent apoptosis (Browne et al., Mol. Cell Biol. 19:8604, 1999).

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Exemplary is thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks. The therapeutic agents may be administered under a metronomic regimen. e.g., continuous low-doses of a therapeutic agent.

Recombinant Constructs

Recombinant constructs are also provided herein and can be used to transform cells in order to express the isolated nucleic acid sequences embodied herein. In certain embodiments, a recombinant nucleic acid construct comprises promoter operably linked to a regulatory region suitable for expressing at least one coronavirus protein. In certain embodiments, the coronavirus is a SARS family of coronaviruses, including severe acute respiratory syndrome coronavirus (SARS-CoV-2) and SARS-OC43 (common cold virus).

In certain embodiments, the recombinant nucleic acid construct comprises promoter operably linked to a regulatory region suitable for expressing at least one SARS-CoV-2 protein comprising a spike (S) protein, a membrane (M) protein, an envelope (E) protein or a nucleocapsid (N) protein or fragments and combinations thereof. In certain embodiments, the one or more coronavirus proteins expressed by the recombinant vector comprise one or more amino acid substitutions, insertions, deletions, mutations, modifications and combinations thereof.

It will be appreciated that a number of nucleic acids can encode a polypeptide having a particular amino acid sequence. The degeneracy of the genetic code is well known in the art. For many amino acids, there is more than one nucleotide triplet that serves as the codon for the amino acid. For example, codons in the coding sequence for S protein can be modified such that optimal expression in a particular organism is obtained, using appropriate codon bias tables for that organism.

Nucleic acids as described herein may be contained in vectors. Vectors can include, for example, origins of replication, scaffold attachment regions, and/or markers. Several delivery methods may be utilized for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005). An example non-viral delivery method may utilize nanoparticle technology. This platform has demonstrated utility as a pharmaceutical in vivo. Nanotechnology has improved transcytosis of drugs across tight epithelial and endothelial barriers. It offers targeted delivery of its payload to cells and tissues in a specific manner (Allen and Cullis, Science, 303:1818-1822, 1998).

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; Bio Techniques, 34: 167-171 (2003). A large variety of such vectors is known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex 1 virus (HSV) vector [Geller, A. I. et al., J. Neurochem, 64: 487 (1995); Lim, F., et al., in DNA Cloning: Mammalian Systems. D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

The polynucleotides disclosed herein may be used with a microdelivery vehicle such as cationic liposomes and adenoviral vectors. For a review of the procedures for liposome preparation, targeting and delivery of contents, see Mannino and Gould-Fogcritc, BioTechniques, 6:682 (1988). See also, Felgner and Holm. Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992). Another delivery method is to use single stranded DNA producing vectors which can produce the expressed products intracellularly. See for example, Chen et al, BioTechniques. 34: 167-171 (2003), which is incorporated herein, by reference, in its entirety.

In certain embodiments of the disclosure, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

The anti-retroviral agents and/or the recombinant constructs of the disclosure can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression.

In some embodiments, the compositions of the disclosure can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI. In some embodiments, the compositions can be formulated as a nanoparticle encapsulating the compositions embodied herein. L-PEI has been used to efficiently deliver genes in vivo into a wide range of organs such as lung, brain, pancreas, retina, bladder as well as tumor. L-PEI is able to efficiently condense, stabilize and deliver nucleic acids in vitro and in vivo.

In some embodiments, delivery of vectors can also be mediated by exosomes. Exosomes are lipid nanovesicles released by many cell types. They mediate intercellular communication by transporting nucleic acids and proteins between cells. Exosomes contain RNAs, miRNAs, and proteins derived from the endocytic pathway. They may be taken up by target cells by endocytosis, fusion, or both. Exosomes can be harnessed to deliver nucleic acids to specific target cells.

The expression constructs of the present disclosure can also be delivered by means of nanoclews. Nanoclews are a cocoon-like DNA nanocomposites (Sun, et al., J. Am. Chem. Soc. 2014, 136:14722-14725). They can be loaded with nucleic acids for uptake by target cells and release in target cell cytoplasm. Methods for constructing nanoclews, loading them, and designing release molecules can be found in Sun, et al. (Sun W, et al., J. Am. Chem. Soc. 2014, 136:14722-14725; Sun W, et al., Angew. Chem. Int. Ed. 2015: 12029-12033.)

The nucleic acids and vectors may also be applied to a surface of a device (e.g., a catheter) or contained within a pump, patch, or any other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

Combination or Alternation Therapy

Accordingly, the invention features compositions which include therapeutically effective amounts of pharmaceutical agents which inhibit viral replication and a recombinant construct embodied herein, are administered sequentially or alternately or in conjunction for preventing or treating a subject.

Accordingly, in certain embodiments, the recombinant constructs are administered as combination therapy with antiviral pharmaceutical agents which inhibit viral replication, such as, for example, by inhibiting viral RNA-dependent RNA polymerases. In certain embodiments, the pharmaceutical agents comprise rilpivirine, remdesivir or the combination thereof. In certain embodiments, the antiviral agents can be administered together with at least one recombinant construct editing agent as part of a unitary pharmaceutical composition. Alternatively, each can be administered apart from the other antiviral agents. In this embodiment, the antiviral agents and the at least one at least one recombinant construct are administered substantially simultaneously, i.e. the compounds are administered at the same time or one after the other, so long as the compounds reach therapeutic levels for a period of time in the blood. In other embodiments, the antiviral agents are administered in one or more doses over a period of time followed by administration of the recombinant construct embodied herein. In certain embodiments, an antiviral agent comprises viral entry inhibitors, reverse transcriptase inhibitors, protease inhibitors, and immune-based therapeutic agents.

In certain embodiments, combination therapy comprises administering to the subject a composition comprising: a therapeutically effective amount of one or more of: non-nucleoside reverse transcriptase inhibitors (NNRTI), nucleoside-analogue antiviral agents or a combination thereof. In certain embodiments, an NNRTI comprises: etravirine, efavirenz, nevirapine, rilpivirine, delavirdine, or nevirapine. In embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In certain embodiments, the nucleoside-analogue antiviral agent comprises a therapeutically effective amount of one or more of remdesivir, ribavirin, lamivudine, stavudine or zidoduvine.

In certain embodiments, combination therapy further comprises administering one or more secondary active agents in conjunction with the compositions, e.g. rilpivirine and/or remdesivir, such as, viral entry inhibitors, reverse transcriptase inhibitors, protease inhibitors, and immune-based therapeutic agents.

In general, in combination therapy, effective dosages of two or more agents are administered together, whereas during alternation therapy, an effective dosage of each agent is administered serially. The dosage will depend on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

Combination therapy may be administered as (a) a single pharmaceutical composition which comprises an antiviral agent as described herein, at least one or more secondary active agents as described herein, and a pharmaceutically acceptable excipient, diluent, or carrier; or (b) two separate pharmaceutical compositions comprising (i) a first composition comprising an anti-retroviral agent as embodied herein and a pharmaceutically acceptable excipient, diluent, or carrier, and (ii) a second composition comprising at least one or more secondary active agents as embodied herein. The pharmaceutical compositions can be administered simultaneously or sequentially and in any order.

The antiviral agents may be a nucleoside reverse transcriptase inhibitor, a nucleotide reverse transcriptase inhibitor, a non-nucleoside reverse transcriptase inhibitor, a protease inhibitor, an integrase inhibitor, a fusion inhibitor, a maturation inhibitor, or a combination thereof.

In certain embodiments, the at least one antiviral agent comprises: myristolyated dolutegravir, lamivudine, abacavir, rilpivirine or combinations thereof.

In certain embodiments, a composition comprises a therapeutically effective amount of a non-nucleoside reverse transcriptase inhibitor (NNRTT) and/or a nucleoside reverse transcriptase inhibitor (NRTI), and/or myristolyated dolutegravir, lamivudine, abacavir, rilpivirine analogs, variants or combinations thereof. In certain embodiments, an NNRTI comprises: etravirine, efavirenz, nevirapine, rilpivirine, delavirdine, or nevirapine.

In embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof.

In certain embodiments, the composition further comprises at least one or more protease inhibitors. In certain embodiments, a protease inhibitor comprises: amprenavir, tipranavir, indinavir, saquinavir mesylate, lopinavir and ritonavir (LPV/RTV), Fosamprenavir Calcium (FOS-APV), ritonavir, darunavir, atazanavir sulfate, nelfinavir mesylate or combinations thereof.

In addition, one or more agents which alleviate any other symptoms that may be associated with the virus infection, e.g. fever, chills, headaches, secondary infections, can be administered in concert with, or as part of the pharmaceutical composition or at separate times. These agents comprise, without limitation, an anti-pyretic agent, anti-inflammatory agent, chemotherapeutic agent, or combinations thereof.

Some antiviral agents which can be used for combination therapy include agents that interfere with the ability of a virus to infiltrate a target cell. The virus must go through a sequence of steps to do this, beginning with binding to a specific “receptor” molecule on the surface of the host cell and ending with the virus “uncoating” inside the cell and releasing its contents. Viruses that have a lipid envelope must also fuse their envelope with the target cell, or with a vesicle that transports them into the cell, before they can uncoat.

There are two types of active agents which inhibit this stage of viral replication. One type includes agents which mimic the virus-associated protein (VAP) and bind to the cellular receptors, including VAP anti-idiotypic antibodies, natural ligands of the receptor and anti-receptor antibodies, receptor anti-idiotypic antibodies, extraneous receptor and synthetic receptor mimics. The other type includes agents which inhibit viral entry, for example, when the virus attaches to and enters the host cell. Further antiviral agents that can be used include agents which interfere with viral processes that synthesize virus components after a virus invades a cell. Representative agents include nucleotide and nucleoside analogues that look like the building blocks of RNA or DNA, but deactivate the enzymes that synthesize the RNA or DNA once the analogue is incorporated. Acyclovir is a nucleoside analogue, and is effective against herpes virus infections. Zidovudine (AZT), 3TC, FTC, and other nucleoside reverse transcriptase inhibitors (NRTI), as well as non-nucleoside reverse transcriptase inhibitors (NNRTI), can also be used. Integrase inhibitors can also be used.

Still other active agents function by stimulating the patient's immune system. Interferons, including pegylated interferons, are representative compounds of this class.

In certain embodiments, the antiviral agent comprises therapeutically effective amounts of: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines or combinations thereof.

The compositions described herein are suitable for use in a variety of drug delivery systems described above. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue specific antibody. The liposomes will be targeted to and taken up selectively by the organ.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder.

When treating viral infections, an effective amount of the inhibitory compound is an amount sufficient to suppress the growth and proliferation of the virus. Viral infections can be prevented, cither initially, or from re-occurring, by administering the compounds described herein in a prophylactic manner. Preferably, the effective amount is sufficient to obtain the desired result, but insufficient to cause appreciable side effects.

Pharmaceutical Agents

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

The compositions described herein are suitable for use in a variety of drug delivery systems. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue specific antibody. The liposomes will be targeted to and taken up selectively by the organ.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder.

When treating viral infections, an effective amount of the inhibitory compound is an amount sufficient to suppress the growth and proliferation of the virus. Viral infections can be prevented, either initially, or from re-occurring, by administering the compounds described herein in a prophylactic manner. Preferably, the effective amount is sufficient to obtain the desired result, but insufficient to cause appreciable side effects.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the disclosure, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the disclosure can include a single treatment or a series of treatments.

The effective dose can vary, depending upon factors such as the condition of the patient, the severity of the viral infection, and the manner in which the pharmaceutical composition is administered. The effective dose of compounds will of course differ from patient to patient, but in general includes amounts starting where desired therapeutic effects occur but below the amount where significant side effects are observed. For human patients, the effective dose of typical compounds generally requires administering the compound in an amount of at least about 1, often at least about 10, and frequently at least about 25 μg/24 hr/patient. The effective dose generally does not exceed about 500, often does not exceed about 400, and frequently does not exceed about 300 μg/24 hr/patient. In addition, administration of the effective dose is such that the concentration of the compound within the plasma of the patient normally does not exceed 500 ng/mL and frequently does not exceed 100 ng/mL.

A subject is effectively treated whenever a clinically beneficial result ensues. This may mean, for example, a complete resolution of the symptoms of a disease, a decrease in the severity of the symptoms of the disease, or a slowing of the disease's progression. These methods can further include the steps of a) identifying a subject (e.g., a patient and, more specifically, a human patient) who has a certain disease to be treated; and b) providing to the subject the compositions comprising at least one anti-viral and/or a secondary active agent embodied herein.

In certain embodiments, the pharmaceutical compositions are specifically adapted for topical administration to the nasal cavity. The pharmaceutical compositions may be applied before or after the outbreak of a respiratory viral infection in a human individual. Even if applied after the outbreak of a viral infection the compositions still prevent or at least ameliorate late complications of respiratory viral infections. Such complications are known in the art and include—but are not limited to—complications in connection with secondary infections by bacteria, and deterioration of pre-existing diseases such as allergy or COPD.

Topically administrable intranasal compositions referred to herein may have a pH value within a range of from 3.5 to 8.0, usually within a range of from about 4.0 to about 8.0. They may comprise one or more nasally compatible pH adjusting agents or buffer systems that prevent pH drift during storage. Such pH adjusting agents include, but are not limited to, boric acid, sodium borate, potassium citrate, citric acid, sodium bicarbonate, and various inorganic phosphate buffers such as Na₂HPO₄, NaH₂PO₄, KH₂PO₄, and mixtures thereof. The minimal ionic strengths introduced by any such pH-adjusting agents do not interfere with the essence of the disclosure. To prevent precipitation of calcium with phosphate ions from the buffer system, EDTA may be added up to a concentration of 2 mg/ml. In addition, flavors such as Eucalyptus, campher, menthol, peppermint or similar, by way of oils or extracts, may be added to the product at concentrations known in the art.

Also, the topical intranasal formulations referred to herein may comprise one or more intranasally compatible surfactants. The surfactant facilitates the spread of the formulation across the surface of the nasal mucosa and may be non-ionic or anionic. Exemplary non-ionic surfactants may be selected from the group comprising tyloxapol, polyoxyethylene sorbitan esters, polyethoxylated castor oils, poloxamers, polyoxyethylene/polyoxypropylene surfactants, polyoxyethylene stearate, polyoxyethylene propylene glycol stearate, hydroxyalkylphosphonate, lauric or palmitic acid esters and ethers, triethanol amine oleate, or from a combination of the foregoing agents. Still further suitable surfactants may be known to those skilled in the art. The surfactants may typically be present at concentrations of from 0.02% (w/v) to 0.1% (w/v) of the composition.

In various embodiments, the present topical intranasal preparation may contain one or more preservatives to inhibit microbial growth and to prolong shelf life. Exemplary preservatives include, but are not limited to, disodium edetate (EDTA) and potassium sorbate. The preservative amount is typically less than about 0.02% (w/v) of the total composition. EDTA may be added up to 2 mg/ml.

In addition to the ingredients mentioned above, it is contemplated that a variety of additional or alternative ingredients may be present in the pharmaceutical compositions of the present disclosure, which additional or alternative ingredients include anti-oxidants such as vitamin E or its commercially available derivatives such as tocopherol polyethylene glycol 1000 succinate (TPGS), ascorbic acid, or sodium metabisulfite.

The pharmaceutical compositions herein are typically provided in sterile form for topical administration to the nasal cavity and are preferably adjusted for self-administration by the individual in need thereof. In one embodiment, the preparation is a particle-free nasal spray. Other suitable formulations include intranasally acceptable swabs, as well as ointments and gels that can be applied to the nose, optionally as sprays or aerosols.

Pharmaceutical compositions described above can be delivered via different methodologies including sprays, irrigation systems (e.g. netipot), syringes or others. The composition may be provided in a dosage form that is suitable for a nasal aerosol or inhalation administration route. An exemplary method of administration of the composition can include spraying vaporized or nebulized disseminated microparticles under an active dynamic pressure.

Suitable aerosol dispensers for use will be apparent to those skilled in the art and may vary from simple devices analogous to perfume dispensers to pressurized spray cans and even complex apparatus such as might be used in hospitals. Whichever device is used it is generally preferable that it comprises some kind of dosimeter to control the amount of solution administered in one go. One device, which corresponds to a dispenser with a nozzle, effectively incorporates such a dosimeter without any specialized adaptation being necessary, the limit stop of the depressible spray head fixing the maximum single amount of solution dispensable at once. Specially developed spray devices may be made with a hand-held device comprising a reservoir of the composition.

Suitable means for dispersing the spray, preferably in aerosol form, are provided. Examples include pneumatically pressurized devices and devices employing pressurized gas forced across the opening of a tube leading into the reservoir to create an aerosol, and press-button type devices wherein the button, when pressed, creates pressure on the surface of the liquid in the reservoir, forcing it up through a tube and through a fine nozzle to disperse the solution into an aerosol spray. Other examples include aerosol dispensers, inhalers, pump sprayers, nebulizers (such as positive pressure nebulizers), and the like. In some embodiments, the device used is pre-filled with a composition described herein.

One embodiment would include a multi-dose metered dose spray pump allowing for spraying of a fixed volume of solution. Alternatively, gas driven (e.g. nitrogen) devices, such as systems that hold the compositions separate from the propellant in aluminum or plastic (or any other type of) bottle. These devices deliver solution at variable diffusion flows and angles when combined with different actuators. Preferred diffusion flows could deliver 0.5-10 ml solution per spraying second at angles of 0-60°.

The compositions described above can be administered as per physician's instructions and depending on the condition. A preferred mode of (nasal) administration comprises 1-5 sprays per nostril, 1-5 times daily; this could extend to many weeks depending on the condition or symptom to be treated (e.g. in allergy).

The therapeutic methods described herein (that include prophylactic treatment) in general comprise administration of a therapeutically effective amount of the compositions herein to a subject (e.g., animal, human) in need thereof, including a mammal, particularly a human. Such treatment will be suitably administered to subjects, particularly humans, suffering from, having, susceptible to, or at risk for infection by respiratory tract viruses or symptoms thereof. Determination of those subjects “at risk” can be made by any objective or subjective determination by a diagnostic test or opinion of a subject or health care provider.

Kits

The compositions described herein can be packaged in suitable containers labeled, for example, for use as a therapy to treat a subject having a viral infection, for example, SARS-CoV-2 infection or a subject at risk of contracting for example, a SARS-CoV-2 infection. The containers can include a composition comprising at least one anti-viral or anti-retroviral agent; a gene-editing agent and one or more of a suitable stabilizer, carrier molecule, flavoring, and/or the like, as appropriate for the intended use. In other embodiments, the kit further comprises one or more therapeutic reagents that alleviate some of the symptoms or secondary bacterial infections. Accordingly, packaged products (e.g., sterile containers containing one or more of the compositions described herein and packaged for storage, shipment, or sale at concentrated or ready-to-use concentrations) and kits, including at least one composition of the invention, and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and, or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

### EXAMPLES

### Example 1: Pharmacologic Approach for Suppression SARS

Treatment of cells susceptible to infection by SARS-OC43 with rilpivirine decreased viral replication. The next experiments conducted was using a combination of pharmacological agents e.g., rilpivirine and remdesivir to determine a therapeutic and/or protective strategy toward the SARS family of coronaviruses, including SARS-CoV-2 and SARS-OC43 (common cold virus).

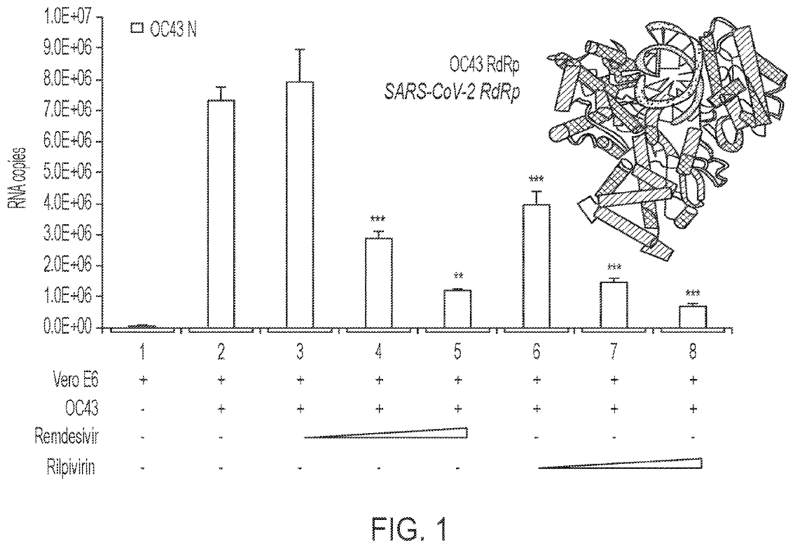

The results obtained demonstrated that Remdesivir and Rilpivirine suppressed human coronavirus OC43 replication in a dose dependent manner (FIG. 1). Vero E6 cells were infected with OC43 (MOI:1). Cells were treated with increasing concentrations of Remdesivir and Rilpivirine (0.1, 1.0, and 10 μM). At 48 hrs post-infections, OC43 RNA copies in the growth media were analyzed by real time qRT-PCR with nucleocapcid (N) primers and shown as bar graph. Data were mean t SEM of three replicates. “P” values were calculated in comparison with control infections (untreated). (Inset) Superimposition of OC43 (model) and SARS-CoV-2 (cryo-EM) RdRp structures.

### Other Embodiments

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

What is claimed:

1. A method of preventing or treating a coronavirus infection, comprising:
administering to a subject in need of such treatment a composition comprising a therapeutically effective amount of one or more anti-viral agents,
thereby treating the subject.

2. The method of claim 1, wherein the composition comprises a therapeutically effective amount of one or more of: non-nucleoside reverse transcriptase inhibitors (NNRTI), nucleoside-analogue antiviral agents or a combination thereof.

3. The method of claim 2, wherein the NNRTI comprises a therapeutically effective amount of one or more of: etravirine, efavirenz, nevirapine, rilpivirine, delavirdine, or nevirapine.

4. The method of claim 3, wherein the NNRTI is rilpivirine.

5. The method of claim 2, wherein the nucleoside-analogue antiviral agent comprises a therapeutically effective amount of one or more of remdesivir, ribavirin, lamivudine, stavudine or zidoduvine.

6. The method of claim 5, wherein the nucleoside-analogue antiviral agent is remdesivir.

7. The method of claim 2, wherein the composition comprises a therapeutically effective amount of rilpivirine and remdesivir.

8. The method of claim 2, wherein the one or more anti-viral agents are co-administered.

9. The method of claim 1, wherein the coronavirus comprises severe acute respiratory syndrome coronavirus (SARS-CoV-2), SARS-OC43, middle east respiratory syndrome coronavirus (MERS-CoV), human coronavirus (HCoV)-OC43, HCoV-229E, HCoV-HKU1 or HCoV-NL63.

10. A pharmaceutical composition comprising a therapeutically effective amount of one or more of: non-nucleoside reverse transcriptase inhibitors (NNRTI), nucleoside-analogue antiviral agents or a combination thereof.

11. The pharmaceutical composition of claim 10, wherein the NNRTI comprises a therapeutically effective amount of one or more of: etravirine, efavirenz, nevirapine, rilpivirine, delavirdine, or nevirapine.

12. The pharmaceutical composition of claim 10, wherein the nucleoside-analogue antiviral agent comprises a therapeutically effective amount of one or more of remdesivir, ribavirin, lamivudine, stavudine or zidoduvine.

13. The pharmaceutical composition of claim 11, wherein the NNRTI is rilpivirine.

14. The pharmaceutical composition of claim 11, wherein the nucleoside-analogue antiviral agent is remdesivir.

15. The pharmaceutical composition of claim 10, wherein the composition comprises a therapeutically effective amount of rilpivirine and remdesivir.

16. A method of preventing or treating a coronavirus infection, comprising:
administering to a subject in need of such treatment a composition comprising a therapeutically effective amount of one or more anti-viral agents, and,
a recombinant vector comprising a nucleic acid sequence encoding a coronavirus protein comprising a spike (S) protein, a membrane (M) protein, an envelope (E) protein or a nucleocapsid (N) protein or immunogenic fragments thereof,
thereby treating the subject.

17. The method of claim 16, wherein the wherein the composition comprises a therapeutically effective amount of one or more of: non-nucleoside reverse transcriptase inhibitors (NNRTI), nucleoside-analogue antiviral agents or a combination thereof.

18. The method of claim 17, wherein the NNRTI comprises a therapeutically effective amount of one or more of: etravirine, efavirenz, nevirapine, rilpivirine, delavirdine, or nevirapine.

19. The method of claim 18, wherein the NNRTI is rilpivirine.

20. The method of claim 17, wherein the nucleoside-analogue antiviral agent comprises a therapeutically effective amount of one or more of remdesivir, ribavirin, lamivudine, stavudine or zidoduvine.

21. The method of claim 20, wherein the nucleoside-analogue antiviral agent is remdesivir.

22. The method of claim 20, wherein the composition comprises a therapeutically effective amount of rilpivirine and remdesivir.
